(19) United States
(12) Patent Application Publication
(10) Pub. No.: US 2006/0223068 A1
(43) Pub. Date: Oct. 5, 2006

(54) Sorting of Carbon nanotubes through selective DNA delamination of DNA/Carbon nanotube hybrid structures

(76) Inventors: Yuegang Zhang, Cupertino, CA (US); Robert J. Chen, Santa Clara, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/095,414

(22) Filed: Mar. 30, 2005

(51) Int. Cl. C12Q 1/68; C12M 1/34

(52) U.S. Cl. 435/6; 435/287.2; 977/924

(57) ABSTRACT

A method is described that involves creating bundles of semi-conducting CNTs by passing electromagnetic radiation through a solution containing individual DNA/metallic CNT hybrid structures and individual DNA/semi-conducting CNT hybrid structures. The method also involves segregating the bundles of semi-conducting CNTs from the DNA/metallic CNT hybrid structures. Another method is described for targeting for selection CNTs of specific diameter and chirality from a solution containing DNA/CNT hybrid structures.

### FIELD OF ART

The field of art relates generally to Carbon nanotubes (CNTs); more specifically, techniques for sorting CNTs of different electronic types.

### BACKGROUND

A Carbon nanotube (CNT) can be viewed as a sheet of Carbon that has been rolled into the shape of a tube. CNTs having certain properties (e.g., a “metallic” CNT having electronic properties akin to a metal) may be appropriate for certain applications while CNTs having certain other properties (e.g., a “semiconducting” CNT having electronic properties akin to a semiconductor) may be appropriate for certain other applications. CNT properties tend to be a function of the CNT's “chirality” and diameter. The chirality of a CNT characterizes its arrangement of carbon atoms (e.g., arm chair, zigzag, helical/chiral). The diameter of a CNT is the span across a cross section of the tube.

Because the properties of a CNT can be a function of the CNT's chirality and diameter, the suitably of a particular CNT for a particular application is apt to depend on the chirality and diameter of the CNT. Unfortunately, current CNT manufacturing processes are only capable of manufacturing batches of CNTs whose tube diameters and chiralities are widely varied. The problem therefore arises of not being able to collect CNTs (e.g., for a particular application) whose diameter and chiralities reside only within a narrow range (or ranges of) those that have been manufactured.

CNTs are also known to have poor solubility. Here, owing to van der Waals forces (it is believed), individual CNTs tend to “bundle together” into groups. Thus, when a batch of manufactured CNTs are made to flow in a fluidic stream (such as an aqueous solution), bundled groups of CNTs are observed drifting/flowing through the liquid together.

Success at improving the solubility of CNTs has been reported. For example, Zheng et al. (“DNA-Assisted Dispersion and Separation of Carbon Nanotubes”, Nature Materials 2, pgs. 338-342, 2003) has published a process by which single stranded DNA (ss-DNA) is used to “break-down” a CNT bundle into individual CNTs wrapped in a helical structure of DNA. Here, a CNT that has bonded in some fashion with DNA so as to form a combined structure of DNA and the CNT is referred to as “DNA/CNT hybrid structure”. A DNA/CNT hybrid structure of DNA and a metallic CNT may be referred to as “DNA/metallic CNT hybrid structure”. A DNA/CNT hybrid structure of DNA and a semi-conducting CNT may be referred to as “DNA/semi-conducting CNT hybrid structure”.

According to the technique taught by Zheng et al., an aqueous solution containing bundles of CNTs is subjected to the presence of ss-DNA. Because the binding energy associated with the coupling of ss-DNA to a CNT is comparable to the binding energy associated with the coupling of CNTs to one other, the application of sonic energy to the solution can create dynamic situations in which an individual CNT that is bundled with one or more other CNTs will reach a lower energy state if the CNT binds with ss-DNA molecules instead of the CNTs associated with its bundle. Because physical systems tend to fall to lower energy states, this prompts the formation of an individual (i.e., non bundled) CNT helically wrapped in ss-DNA. That is, the CNT essentially leaves its bundle in favor of being helically wrapped by ss-DNA.

According to follow-up work reported by Zheng et al. in “Structure-Based Carbon Nanotube Sorting by Sequence Dependent DNA Assembly”, Science 28 Nov. 2003; 302: 1545-1548, a particular sequence of ss-DNA can be made to self assemble into a helical structure that wraps around the surface of an individual CNT. Individual CNTs wrapped by ss-DNA can then be sorted according to their electrical characteristics through anion exchange chromatography. In this manner, individual CNTs having specific “sought-for” electrical characteristics can be collected.

### FIGURES

The present invention is illustrated by way of example, and not limitation, in the figures of the accompanying drawings in which like references indicate similar elements and in which:

FIG. 1 shows a method for sorting select CNTs and non-select CNTs;

FIG. 2 shows a depiction of a method that is consistent with the method of FIG. 1;

FIG. 3 shows a method for creating bundles of CNTs;

FIG. 4 shows a depiction of a method that is consistent with the method of FIG. 3;

FIG. 5a (prior art) shows a density of states diagram for a metallic CNT;

FIG. 5b (prior art) shows a density of states diagram for a semi-conducting CNT;

FIG. 6 relates to an example for setting radiation photonic energies;

FIG. 7 shows equipment that can perform the methods of FIGS. 1 and 3.

### DESCRIPTION

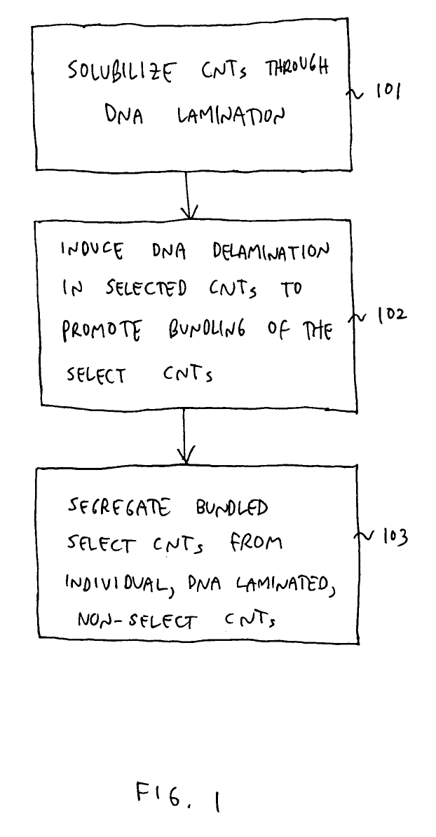

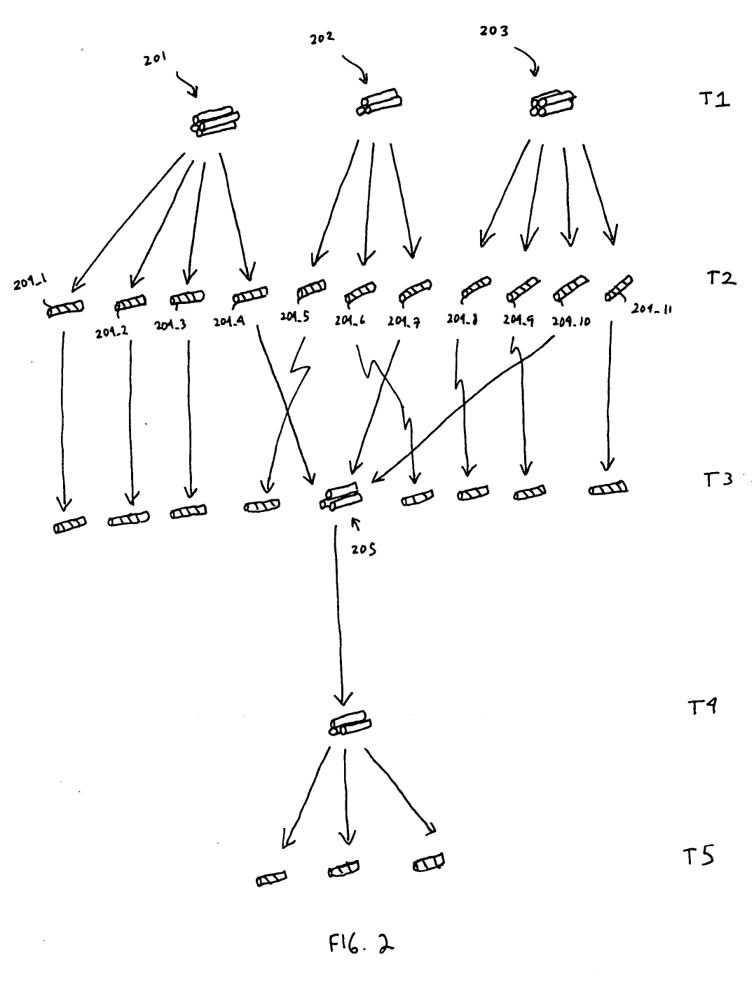

FIGS. 1 and 2 show a CNT sorting technique that is aimed at selecting CNTs of a certain electronic property type (e.g., semi-conducting) from a solution containing individual, DNA wrapped CNTs of various electronic types (e.g., a mixture of metallic, semi-conducting, and semi-metallic CNTs). The sorting technique of FIG. 1 is founded on the ability to “delaminate” 102 the DNA wrapping from “select” CNTs having a specific electronic property type (e.g., semi-conducting CNTs). By substantially removing the DNA wrapping from the select CNTs, the select CNTs are permitted to bundle with one another. The bundles are then segregated from the other “non-select” CNTs that are not of the specific electronic property type and therefore retained their DNA wrapping.

Referring to FIGS. 1 and 2 in detail, initially as depicted at time “T1” in FIG. 2, within a solution containing bundles 201, 202, 203 of CNTs having diverse electronic property types, a DNA lamination process is executed 301 so that the bundles 201, 202, 203 of CNTs are “broken-down” 101 into individual CNTs 204_1 through 204_10 wrapped in DNA. FIG. 2 shows the breakup of bundles 201, 202, 203 into individual CNTs 204_1 through 204_10 between times T1 and T2.

According to one implementation, single walled Carbon nanotubes (SWNTs) manufactured according to a High Pressure Carbon Monoxide (HiPCO) process by Carbon Nanotechnolgies Inc. are solubilized in an aqueous solution with the aid of a custom-synthesized oligonucleotide supplied by Operon Biotechnologies, Inc. The custom-synthesized oligonucleotide has a sequence of TTTTTTTTTTTTTTTTTTTTTTTTTTTTTT (PolyT30). In a typical preparation, the HiPco SWNTs (˜1 mg) are combined with a stock solution of PolyT30 (˜3 ml, 200 μM in 18 MΩ water) and ultrasonicated in an ice water bath (VWR 75D bath sonicator) for 2-3 hrs. at a power of 90 W with frequent vortexing interspersed throughout.

The solution is then centrifuged at 16,000×g for 90 min. and the supernatant decanted and collected. This is repeated times, then the resulting supernatant is dialyzed (MWCO 60,000) against pure water for a period of 2-3 days to remove any free DNA not coated directly on the SWNTs. This procedure typically yields a final concentration of 0.1-0.2 mg/ml of solubilized SWNTs.

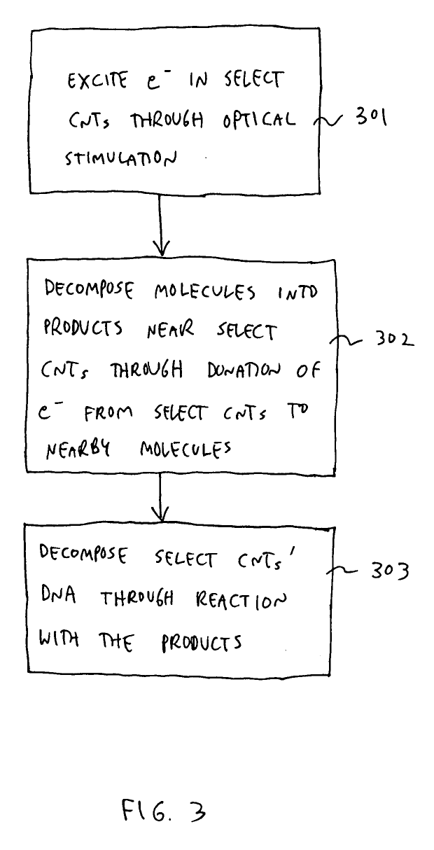

Once the solution is essentially solubilized 101 (i.e., bundles of CNTs are broken down into individual CNTs), the DNA wrapping is substantially removed 102 only from certain “select” CNTs having a specific type of electronic property (e.g., metallic or semi-conducting). In FIG. 2, CNTs 204_4, 204_7 and 204_10 are presumed to be the select CNTs. All other CNTs (i.e., CNTS 204_1 through 204_3, 204_5, 204_6, 204_8, 204_9 and 204_11) are non select CNTs. A process that selectively performs DNA delamination 102 only for CNTs of a specific electronic property type is discussed in more detail further below with respect to FIGS. 3, 4 and 5.

With DNA being delaminated only from the select CNTs 204_4, 204_7 and 204_10, the solution will be in a state that promotes the bundling of the select CNTs with one another. That is, bundles will be created whose constituent CNTs are only the select CNTs. FIG. 2 shows the bundling of select CNTs 204_4, 204_7 and 204_10 into bundle 205 between times T2 and T3. Here, it can be assumed that the delaminating and bundling processes of the select CNTs 204_4, 204_7 and 204_10 occur between times T2 and T3.

Once bundles of the select CNTs are formed, such bundles can be readily segregated from the individual non select CNTs. For example, because of their larger size and weight, the bundled, select CNTs could be precipitated 103 from the individual, non select CNTs through differential centrifugation. FIG. 2 attempts to indicate successful segregation at time T4, where, bundle 205 is observed “alone”. After segregation of the select CNT bundles, the select CNT bundles may then be subjected to a second DNA lamination process so as to put the select CNTs back in individual form. FIG. 2 shows this process between times T4 and T5.

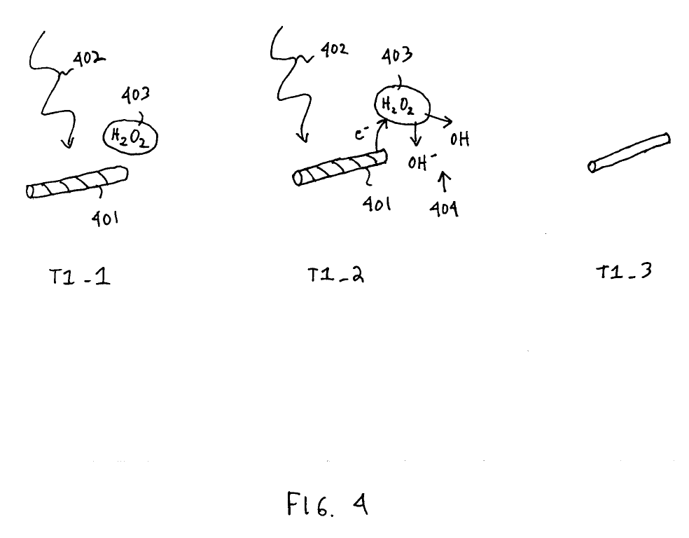

FIGS. 3, 4 and 5 relate to a process for selectively delaminating DNA wrapped CNTs such that only CNTs of a specific electronic property type have their DNA wrapping removed. According to the flow diagram of FIG. 3 and the schematic depiction of FIG. 4, electromagnetic radiation (e.g., light from a laser) is passed through 301 a solution containing: 1) individual, DNA wrapped CNTs of various electronic property types; and, 2) molecules that can be broken down into one or more products that can react with a CNT's DNA wrapping so as to cause the decomposition of the DNA wrapping. At time T1_1 in FIG. 4, a particular DNA wrapped CNT 401 is shown being radiated with light 402. Nearby is a molecule of hydrogen peroxide (H₂O₂) 403.

As will be described in more detail further below, in an ideal environment, the photon energy of the electromagnetic radiation 402 (which hereafter will be referred to as “light” for simplicity) is approximately equal to that of a energy difference between van Hove singularities within the select CNTs so that only electrons within the solution's select CNTs are sufficiently excited (in response to the irradiation by the light) to jump to a higher energy band and then transfer to a nearby molecule. In short, select CNTs are meant to behave as electron donors to the molecules that are near to them; whereas, non-select CNTs are not meant to behave as electron donors to the molecules that are near to them.

Accordingly, part of the behavior that is intended to be induced within the solution is the break down of a molecule caused by the molecule's reception of an electron that was donated by a select CNT 302. For example, in the case of the hydrogen peroxide molecule, and as shown at time T1_2 in FIG. 4, the reception of an electron donated by CNT 401 to the hydrogen peroxide molecule 403 causes the hydrogen peroxide to be broken down into an electrically neutral hydroxyl radical (OH) and an ionic hydroxyl radical (OH⁻). Ideally, as described just above, the electron received by the molecule 403 is donated by a select CNT rather than a non select CNT. Thus, CNT 401 of FIG. 4 is supposed to be a select CNT rather than a non select CNT.

One or more of the products produced by the molecule's breakdown are expected to be able to react with a CNT's DNA wrapping so as to decompose the DNA wrapping. Better said, the products produced by the molecule's breakdown are able to essentially “attack” a CNT's DNA wrapping. In the case of the hydroxylproducts, hydroxylproducts are believed to be able to cause single strand breaks and/or dual strand breaks in DNA chains. As such, it is believed that hydroxylproducts are able to successfully fragment the DNA wrapping around a CNT.

The probability of an electron being successfully transferred from a donating CNT to a receiving molecule greatly reduces as the distance between the donating CNT and the receiving molecule grows. Thus, for the most part, only molecules that are “nearby” a donating CNT are expected to be broken down. In an ideal environment, as discussed above, only select CNTs donate electrons to their nearby molecules and all non-select CNTs do not donate any electrons to their nearby molecules. Therefore, in such an ideal environment, only molecules near select CNTs will be broken down.

As a result, particularly for products having a short lifetime (such as hydroxylproducts in an aqueous solution), the CNT closest to the products of a broken down molecule should be the CNT that donated the electron which caused the molecule's break down. Therefore, in an ideal environment in which only select CNTs donate electrons, for the most part, select CNTs will have their DNA wrappings fragmented from reactions with products that result from molecular breakdown. FIG. 4 therefore shows select CNT 401 as having its DNA wrapping removed at time T1_3. In a more realistic environment, because the behavior outlined just above is apt to be a prevalent or dominant behavior within the solution, over time, selected CNTs are apt to receive substantially more damage to their corresponding DNA wrappings than non selected CNTs.

Regardless, the substantially achieved effect is that the rate at which the DNA wrappings of select CNTs are damaged will be greater than the rate for non-select CNTs. As such, select CNTs will favor bundling with other CNTs substantially earlier than non-select CNTs will (if at all). Therefore, upon the subjection of specially tuned light and appropriate molecules to a solution of DNA solubilized CNTs, bundles of select CNTs are apt to appear in the solution before bundles of non-select CNTs. By ceasing the irradiation of the solution with the light before the appearance of bundles formed with non select CNTs (if any), the solution can be made to produce bundles of only select CNTs. The bundles of select CNTs can then be segregated 303 from the individual non select CNTs (e.g., by precipitation through differential centrifugation).

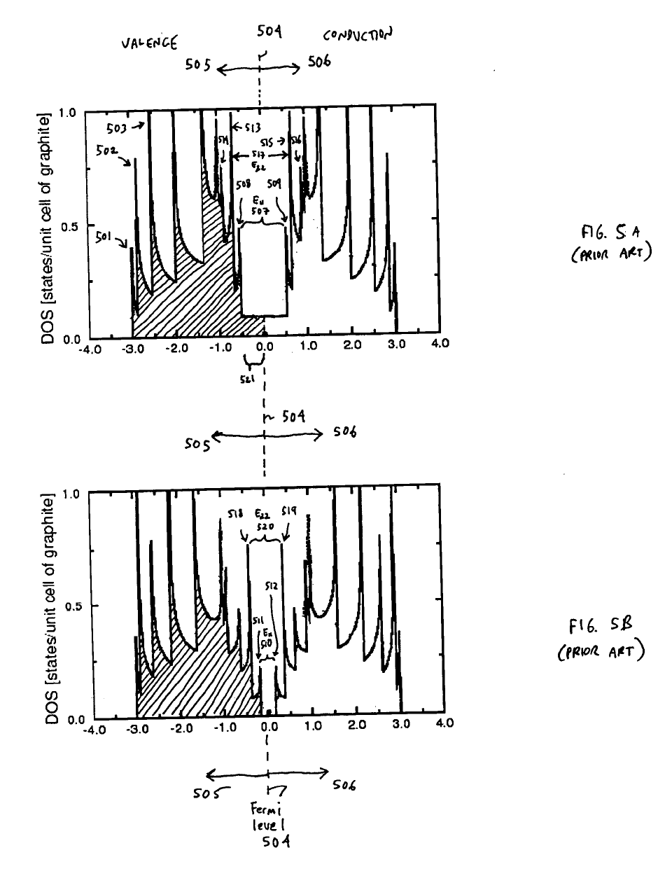

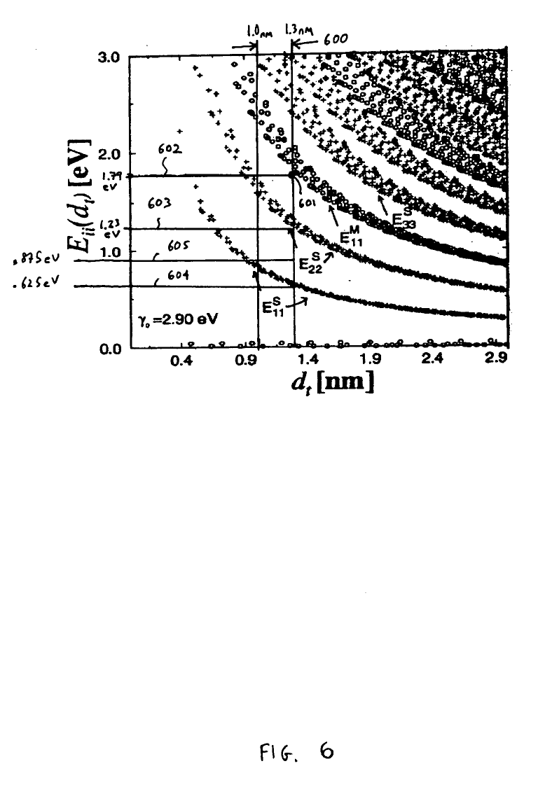

FIGS. 5a, 5b and 6 can be used to describe the relationship between the energy of the incident light and the transfer of donor electrons from select CNTs as opposed to non select CNTs. FIG. 5a shows the “density of states” for an exemplary metallic single walled CNT having a chirality vector of (9,0) and FIG. 5b shows the density of states for an exemplary semi-conducting single walled CNT having a chirality vector of (10,0). Both figures assume a carbon-carbon energy overlap integral γ₀=2.9 eV. Electrons within a CNT must each occupy their own “state” and each state corresponds to a certain amount of electron energy. It is possible for states that are “different” to nevertheless correspond to the same amount of electron energy. The density of states diagrams of FIGS. 5a and 5b each represent “how many” states exist within their respective CNTs across the spectrum of possible electron energies.

A characteristic of CNTs is the presence of van Hove singularities in their density of states vs. energy diagrams. A van Hove singularity appears as a “spike” within a density of states vs. energy diagram. Each of FIGS. 5a and 5b show multiple van Hove singularities (e.g., in FIG. 5a, van Hove singularities are observed at energy levels 501, 502 and 503 as well as a number of other energy levels as well). Also, for both of the density of states diagrams of FIGS. 5a and 5b, the Fermi-level is assumed to be aligned with the 0.0 value 504 on the normalized energy axis.

The Fermi-level 504 is a kind of “water-line” that indicates which energy states are populated with electrons and which energy states are not populated with electrons. Simplistically, under normal conditions, nearly all of the energy states beneath the Fermi-level 504 (which corresponds to region 505 in FIGS. 5a and 5b) are populated with electrons; while, nearly all of the energy states above the Fermi-level 504 (which corresponds to region 506 in FIGS. 5a and 5b) are not populated with electrons. As such, region 505, which is also referred to as the “valence band”, is shaded to indicate the existence of electrons at the corresponding energy levels, and, region 506, which is also referred to as the “conduction band”, is un-shaded to indicate the absence of electrons at the corresponding energy levels.

Referring to the metallic CNT's density of states diagram in FIG. 5a, note that the portion 507 of the diagram between the highest energy van Hove singularity 508 in the valence band 505 and the lowest energy van Hove singularity 509 in the conduction band 506 is continuous and non-zero. By contrast, referring to FIG. 5b, the portion 510 of the density of states diagram for the semi-conducting CNT between the highest energy van Hove singularity 511 in the valence band 505 and the lowest energy van Hove singularity 512 in the conduction band 506 is non-existent. Thus, consistent with traditional definitions of matter, the Fermi-level crosses through an existing electron energy state in the case of a metal (FIG. 5a), but, crosses through an electron energy “gap” in the case of a semi-conductor (FIG. 5b).

For simplicity, valence band van Hove singularities are “counted” in decreasing energy order, and conduction band van Hove singularities are “counted” in increasing energy order. That is, for example, referring to FIG. 5a, singularities 508, 513, 514, etc. are counted as i=1, 2, 3, etc.; and, singularities 509, 515, 516, etc. are counted as i=1, 2, 3, etc. As such, the aforementioned widths of regions 507, 510 are both referred to as the “E₁₁,” energy for their particular CNTs. Likewise, as further examples, the energy width 517 between singularities 513 and 515 in FIG. 5a is referred to as the “E22” energy of the metallic CNT, and, the energy width between singularities 514 and 516 are referred to as the “E33” energy of the metallic CNT. Thus, to restate the analysis of the preceding paragraph, note that the metallic CNT of FIG. 5a exhibits a continuous density of states over the expanse of its E₁₁ energy, while, by contrast, the semi-conducting CNT of FIG. 5b exhibits a “gap” in its density of states over the expanse of its E₁₁ energy.

Another point of distinction with respect to the Eii energies of conducting and semi-conducting CNTs is the size of the energy spans. Typically, the Eii energy of a metallic CNT is greater than the Eii energy of a semi-conducting CNT (at least for lower values of x and CNTs of comparable diameter). A comparison of FIGS. 5a and 5b demonstrate this distinction as the E₁₁ energy 507 of FIG. 5a is greater than the E₁₁ energy 510 of FIG. 5b. As described immediately below, the properties of the density of states diagrams for conducting and semi-conducting CNTs discussed above can be used as a basis for determining an appropriate optical wavelength (or range thereof) that will cause select CNTs (but not non select CNTs) to donate electrons to their nearby molecules.

Here, it should be evident from the discussion above concerning FIGS. 3 and 4 that the effectiveness of the selection technique is related to the number of electrons actually donated by a select CNT as compared to those donated by a non select CNT. Because donated electrons tend to be “high energy” electrons emitted from energy states within a CNT's conduction band 506, the wavelength of the incident light should correspond to an energy that creates large numbers of high energy electrons in the conduction band of the select CNTs but not the non select CNTs.

Here, because a van Hove singularity essentially represents a relative electron energy level that many electrons are permitted to possess, a strategy for achieving the above effect is to use light whose incident energy is aimed at “populating” van Hove singularities of select CNTs but not those of non select CNTs. The diagrams of FIGS. 5a and 5b can be used to demonstrate an example of this approach. According to a specific approach, the semi-conducting CNTs within the solution are deemed to be the select CNTs and the metallic CNTs are deemed to be the non-select CNTs. By setting the optical wavelength such that it corresponds to a photon energy Eₕᵥ that is at or near to the E₁₁ energy of the semi-conducting CNTs but less than one half the E₁₁ energy of the metallic CNTs, a significant number of “high energy” electrons will be excited into the conduction band of the semi-conducting CNTs but not the metallic CNTs.

Consider the CNTs of FIGS. 5a and 5b as an example. Here, the E₁₁ energy 507 of the metallic CNT of FIG. 5a is approximately 1.1 units of energy and the E₁₁ energy 510 of the semi-conducting CNT of FIG. 5b is approximately 0.4 units of energy. If the incident photon energy Eₕᵥ is set to 0.4 units of energy (which is equal to the E₁₁ energy of the semi-conducting CNT but less than one half the E₁₁ energy of the metallic CNT (=1.1/2=0.55 units of energy)), only very few electrons in the metallic CNT will be able to reach the “first” van Hove singularity in the conduction band 509 through photon absorption.

Because of the metallic CNT's large E₁₁ energy 507, the greatest concentrations of electrons in the metallic CNT that could be launched into the conduction band 509 with only 0.4 units of additional energy are those electrons who energies extend over region 521 in the metallic CNT's density of states (i.e., from the Fermi level 504 down toward the “first” van Hove singularity 508 in the valence band 505). Here, very few total electrons have energies in energy region 521 because the metallic CNT's density of states diagram only reaches a value of approximately 0.1 states per unit cell of graphite in this region 521. As such, only few electrons will be excited into the conduction band through photon absorption.

By contrast, the E₁₁ energy 510 of the semi-conducting CNT of FIG. 5b (approximately 0.4 units of energy) is smaller than the metallic CNT's E₁₁ energy 510. Because of this difference, if the incident photon energy Eₕᵥ is set to 0.4 units of energy, large numbers of electrons in the semi-conducting CNT will be able to jump into the conduction band 506 because large numbers of electrons in the first van Hove singularity in the valence band 505 will be able to “jump” into the “first” van Hove singularity in the conduction band 509 through photon absorption. That is, unlike the metallic CNT, the semi-conducting CNT has both large numbers of valence band electrons that can reach the conduction band with the absorption of 0.4 units of energy, and, large numbers of empty states in the conduction band to receive these excited valence electrons. As such the semi-conducting CNT can sustain more excited electrons than the metallic CNT if the incident light is appropriately tuned.

Another approach that can be used alternatively or in combination with the former approach (in which the incident optical energy was set equal to the E₁₁ energy of the semi-conducting CNT) is to tune the incident photon energy to the E₂₂ energy of the semi-conducting CNT. This approach is particularly appropriate if the E₂₂ energy of the semi-conducting CNT is less than the E₁₁ energy of the metallic CNT. Referring again to FIGS. 5a and 5b, note that the E₂₂ energy 520 of the semi-conducting CNT (FIG. 5b) is approximately 0.8 units of energy which is still less than the 1.1 units of E₁₁ energy 507 for the metallic CNT (FIG. 5b). As such, tuning the incident light to an energy of approximately 0.8 units of energy will permit large numbers of electrons to jump from the second van Hove singularity 518 in the valence band 505 to the second van Hove singularity 519 in the conduction band 506.

An incident light energy of 0.8 units of energy will also, like the former example where Eₕᵥ=0.4 units of energy, permit electrons to jump from the first van Hove singularity 505 in the valence band to the first singularity 512 in the conduction band. However, this form of high energy electron generation (i.e., where Eₕᵥ is substantially different than an Eᵢᵢ energy) is apt to be less efficient because phonon energy will need to be given to the CNT carbon sheet in order for an optically stimulated electron to reach a van Hove singularity in the conduction band (energy must be given to the CNT lattice to account for the energy difference between the incident light and the E₁₁ energy of the CNT). For this reason it is also theoretically possible to target metallic CNTs as the select CNTs by setting the photon energy substantially equal to the E₁₁ and/or E₂₂ energies (and/or the Eii energies generally) of the select, metallic CNTs. Further discussion in this regard is provided in more detail further below.

With respect to the metallic CNT, with an incident optical energy of 0.8 units of energy, more electrons will be able to reach the conduction band than the case where the photon energy is only 0.4 units of energy (with at least some of these reaching the first van Hove singularity 509 in the conduction band 506). As such, greater electron transfer from metallic CNTs to nearby molecules is possible. However, the number of high energy electrons generated in the semi-conducting CNT's conduction band will far outweigh those in the metallic CNT's—particularly considering the height of the second singularities 518, 519 in the semi-conducting CNTs against the height of the first singularities 508, 509 in the metallic CNT. Thus, the semi-conducting CNTs are expected to donate substantially more electrons to their nearby molecules than the metallic CNTs.

The analysis above only compared a pair of CNTs. FIG. 6 indicates that the differences between metallic and semi-conducting CNTs elaborated on above with respect to FIGS. 5a and 5b can be extended to large groups of CNTs. Therefore, the approaches described above for causing semi-conducting CNTs to transfer electrons to nearby molecules at greater rates/quantities than metallic CNTs are suitable for segregating semi-conducting and metallic CNTs from one another, consistent with the discussions provided above concerning FIGS. 1 through 4, in a solution containing mixtures of semi-conducting and metallic CNTs having varied chiralities and diameters.

FIG. 6 shows Eii energies for metallic and semi-conducting CNTs as a function of CNT diameter for all known chirality vectors (again, assuming a carbon-carbon energy overlap integral γ₀=2.9 eV). With respect to the labeled trends observed in FIG. 6, a superscript of M denotes a metallic CNT and a superscript of S denotes a semi-conducting CNT. More generally, an “o” is used to plot a specific metallic CNT chirality and diameter; and, a “+” is used to plot a specific semi-conducting CNT chirality and diameter.

From FIG. 6, it is clear that for CNTs of same diameter, the E₁₁ and E₂₂ energies of semi-conducting CNTs are less than the E₁₁ energies of metallic CNTs, irrespective of chirality vector. Owing to the inverse dependence on CNT diameter for these energies, however, some control of CNT diameter variation should be exercised if segregation of metallic and semi-conducting CNTs is attempted through optically stimulated DNA delamination. Better said, because the observed trends are curved, the range of CNT diameters within a solution to be segregated should be managed and limited.

It is presently understood in the art that different CNT manufacturing processes exist, and that, sufficient control can be exercised over these manufacturing processes such that the diameters of a batch of manufactured CNTs will only fall within a limited range (e.g., a HiPCO process gives a typical diameter may yield a diameter range of 0.9 nm-1.3 nm and a modified supported catalyst chemical vapor deposition process may give a diameter range of 0.7 nm-0.9 nm)).

As a simple example, establishment of optical photon energies for segregating semi-conducting CNTs from metallic CNTs produced will be described for an exemplary CNT manufacturing process that exhibits a yielded diameter range of 1.0 to 1.3 nm. This corresponds to a batch of CNTs that fall within region 600 of FIG. 6 and would therefore set an upper corner 601 on the maximum optical energy 602 (1.79 eV) that should be used to screen semi-conducting CNTs from metallic CNTs. Here, consistent with the discussion concerning FIGS. 5a and 5b, if the incident photon energy is kept beneath the E₁₁ energies of all the metallic CNTs within the manufactured sample, all of the metallic CNTs should only be able to weakly donate electrons to their nearby molecules.

If the maximum optical energy 602 is set to 1.79 eV, a minimum optical energy 603 can be set to 1.23 eV. By sweeping the optical wavelength such that the incident photon energy sweeps from level 602 to level 603, in theory from FIG. 6, the solution will be radiated across the E₂₂ energies for all possible manufactured semi-conducting CNTs. If the minimum optical energy level is instead set to 0.625 eV (i.e., level 604), the solution will be radiated across the E₂₂ and E₁₁ energies for all the possible manufactured semi-conducting CNTS. As another approach, if the radiated energy is instead swept across a range of 0.875 eV and 0.625 eV (i.e., levels 605 and 606), the solution will be radiated across the E₁₁ energies for all possible manufactured semi-conducting CNTs. Consistent with the discussions provided above for FIGS. 5a and 5b, this should be sufficient to generate bundles of semi-conducting CNTs while largely leaving the individual DNA/metallic CNT hybrid structures intact.

Although this particular example yielded optical energies whose wavelengths substantially correspond to the infra-red realm, it is important to note that different manufactured diameter ranges (i.e., manufactured diameter ranges other than 1.0-1.3 nm) may yield different optical energy ranges as well.

It is also important to point out that the technique described above can also be used to bundle one or more CNTs of specific diameter and chirality. That is, rather than a “select” CNT simply being a semi-conducting CNT, a “select” CNT may be a CNT of specific chirality and diameter. The ability to cause the DNA delamination of one or more CNTs of specific chirality and diameter derives from the fact that the E₁₁ energy for a specific CNT is determined by that CNT's chirality and diameter.

Thus, for example, with knowledge of the particular E₁₁ and/or E₂₂ energy for a CNT of specific chirality and diameter, the optical photon energy can be set substantially equal to the E₁₁ and/or E₂₂ energy of the CNT so as to cause DNA delamination and bundling of only those CNTs having the specific diameter and chirality. Here, the CNT can be either a semi-conducting or metallic CNT. Of course, it is possible to use energies other than the E₁₁ and/or E₂₂ energies (e.g., E₃₃ energy, etc.).

Also, the process of “targeting” CNTs of specific diameter and chirality as the select CNTs can be repeated for additional CNTs that a collection of different diameter and chiralities correspond to the collect CNTs. For example, CNTs of a first diameter and chirality combination, and, CNTs of a second diameter and chirality combination are extracted by setting optical energies substantially equal to the E₁₁ and/or E₂₂ energies that correspond to the first diameter and chirality combination and the second diameter and chirality combination. Note that various selection strategies could be chosen with respect to what type of CNTs are targeted as the select CNTs. For example, the first chirality and diameter combination could correspond to a semi-conducting CNT, and, the second chirality and diameter combination could correspond to metallic CNT. In other instances all targeted CNTs could be metallic CNTs, or, all targeted CNTs could be semi-conducting CNTs.

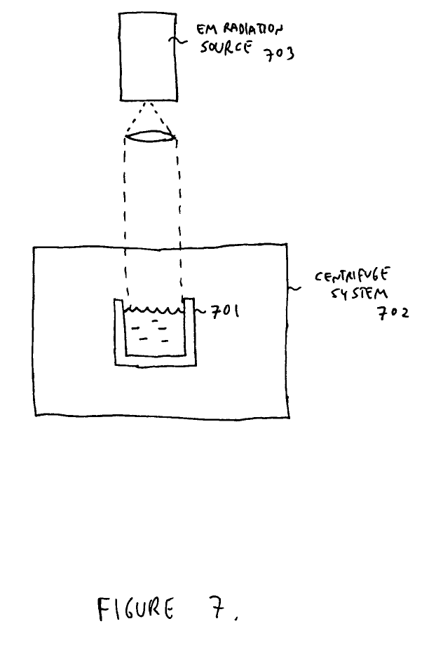

FIG. 7 shows an apparatus for performing the sorting techniques described above. According to the depiction of FIG. 7, there exists a container 701 that holds a solution of DNA wrapped, individual CNTs and a chemical corresponding to the molecules whose products strip away the DNA around select CNTs. An electromagnetic radiation source 703 (e.g., a laser light source) having variable wavelength control is used to irradiate the container 701 and its solution (noting that wavelength is directly related to photon energy)—typically over a range of wavelengths. Once the solution in the contained 701 has been sufficiently radiated by the radiation source 703 to generate a sufficient quantity of bundles of select CNTs, a centrifuge system 702 is used to precipitate out the bundles of select CNTs. In an implementation, optical pieces (e.g., one or more lenses and/or mirrors) that process radiation emitted by the radiation source 703 are arranged to shine the emitted radiation through the container 701 while the container 701 is in the grasp of the centrifuge system 702.

In the foregoing specification, the invention has been described with reference to specific exemplary embodiments thereof. It will, however, be evident that various modifications and changes may be made thereto without departing from the broader spirit and scope of the invention as set forth in the appended claims. The specification and drawings are, accordingly, to be regarded in an illustrative rather than a restrictive sense.

1. A method, comprising:
creating bundles of semi-conducting CNTs by exposing a solution containing individual DNA/metallic CNT hybrid structures and individual DNA/semi-conducting CNT hybrid structures to electromagnetic radiation; and,
segregating said bundles of semi-conducting CNTs from said DNA/metallic CNT hybrid structures.

2. The method of claim 1 wherein said solution contains hydrogen peroxide molecules.

3. The method of claim 1 wherein said electromagnetic radiation has a wavelength that falls within the infrared spectrum.

4. The method of claim 1 wherein said segregating comprises precipitating said bundles with centrifugation.

5. The method of claim 1 further comprising forming a second set of individual DNA/metallic CNT hybrid structures from said bundles.

6. The method of claim 1 wherein photonic energy of said electromagnetic radiation is set substantially equal to an E₁₁ energy of a semi-conducting CNT that is part of one of said DNA/semi-conducting CNT hybrid structures.

7. The method of claim 1 wherein photonic energy of said electromagnetic radiation is set substantially equal to an E₂₂ energy of a semi-conducting CNT that is part of one of said DNA/semi-conducting CNT hybrid structures.

8. The method of claim 1 wherein said electromagnetic radiation's photonic energy is less than an E₁₁ energy of a metallic CNT that is part of one of said DNA/metallic CNT hybrid structures.

9. A method, comprising:
creating a solution comprising:
1) individual DNA/metallic CNT hybrid structures;
2) individual DNA/semi-conducting CNT hybrid structures;
3) molecules made up of products that react with DNA so as to decompose said DNA
creating bundles of semi-conducting CNTs by exposing said solution to electromagnetic radiation to cause at least a portion of said molecules to break up into their products because they received electrons from at least a portion of said DNA/semi-conducting CNT hybrid structures; and,
segregating said bundles of semi-conducting CNTs from said DNA/metallic CNT hybrid structures.

10. The method of claim 9 wherein said molecules comprise hydrogen peroxide molecules.

11. The method of claim 9 wherein said electromagnetic radiation has a wavelength that falls within the infrared spectrum.

12. The method of claim 9 wherein said segregating comprises precipitating said bundles with centrifugation.

13. The method of claim 9 further comprising forming a second set of individual DNA/metallic CNT hybrid structures from said bundles.

14. The method of claim 9 wherein photonic energy of said electromagnetic radiation is set substantially equal to an E₁₁ energy of a semi-conducting CNT that is part of one of said DNA/semi-conducting CNT hybrid structures.

15. The method of claim 9 wherein photonic energy of said electromagnetic radiation is set substantially equal to an E₂₂ energy of a semi-conducting CNT that is part of one of said DNA/semi-conducting CNT hybrid structures.

16. The method of claim 9 wherein said electromagnetic radiation's photonic energy is less than an E₁₁ energy of a metallic CNT that is part of one of said DNA/metallic CNT hybrid structures.

17. An apparatus, comprising:
a) a container to hold a solution, comprising:
1) individual DNA/metallic CNT hybrid structures;
2) individual DNA/semi-conducting CNT hybrid structures;
3) molecules made up of products that react with DNA so as to decompose said DNA;
b) an electromagnetic radiation source and optical pieces to expose said solution to electromagnetic radiation; and,
c) a centrifuge system to segregate bundles of semi-conducting CNTs from said DNA/metallic CNT hybrid structures.

18. The apparatus of claim 17 where said electromagnetic radiation source is a laser light source.

19. The apparatus of claim 17 wherein said electromagnetic radiation source permits variation of said electromagnetic radiation's wavelength.

20. The apparatus of claim 17 where said optical pieces are integrated with said centrifuge system.
